(19) United States
(12) Patent Application Publication
(10) Pub. No.: US 2017/0071901 A1
(43) Pub. Date: Mar. 16, 2017

(54) METHODS AND FORMULATIONS FOR INCREASING CHEMICAL STABILITY AND BIOLOGICAL ACTIVITY OF PHENOLIC COMPOUNDS

(71) Applicants: Guodong Zhang, Amherst, MA (US); Eric Decker, Amherst, MA (US); Yoshiki Nimiya, Amherst, MA (US)

(72) Inventors: Guodong Zhang, Amherst, MA (US); Eric Decker, Amherst, MA (US); Yoshiki Nimiya, Amherst, MA (US)

(21) Appl. No.: 15/259,242

(22) Filed: Sep. 8, 2016

Related U.S. Application Data
(60) Provisional application No. 62/218,684, filed on Sep. 15, 2015.

(51) Int. Cl. A61K 31/353; A61K 47/10; A61K 47/22; A61K 31/12; A61K 9/08

(52) U.S. Cl. CPC A61K 31/353; A61K 31/12; A61K 9/08; A61K 47/22; A61K 47/10

(57) ABSTRACT

The present teachings show that co-addition of a series of redox active antioxidants with diverse chemical structures dramatically increases the chemical stability of dietary phenolic compounds, enhanced their biological activities in cells, and boosted the circulating concentrations of these compounds in animal models.

### CROSS REFERENCE TO RELATED APPLICATIONS

This application claims priority to and benefit of U.S. Provisional Application No. 62/218,684, entitled METHODS AND FORMULATIONS FOR INCREASING CHEMICAL STABILITY AND BIOLOGICAL ACTIVITY OF PHENOLIC COMPOUNDS, filed on Sep. 15, 2015, which is incorporated by reference herein in its entirety and for all purposes.

### BACKGROUND

These teachings relates generally to stabilizing phenolic compounds,

Substantial human and pre-clinical studies have shown that dietary phenolic compounds (such as curcumin) have potent health-promoting effects. Stability of dietary phenolic compounds has been a concern. For example, a problem to develop curcumin-based therapeutics is its poor chemical stability: its half-life at physiological PH is only several minutes. In part due to its rapid degradation, curcumin has a poor pharmacokinetics profile, and high doses of curcumin were needed to exert its biological actions in vivo, Chronic intake of high-dose curcumin could cause some adverse effects and lead to patient withdrawal, limiting its therapeutic applications.

Substantial human and pre-clinical studies have shown that curcumin, a dietary compound from turmeric, has potent anti-cancer, anti-inflammatory and anti-oxidative effects. For example, in terms of cancer, a Phase IIα human clinical trial of colorectal cancer showed that daily intake of 96 4 grams of curcumin for a month caused a ˜40% reduction of aberrant crypt foci. In another Phase II human clinical trial, daily intake of 8 grams of curcumin demonstrates anti-cancer efficacy in some patients with advanced pancreatic cancer. Currently the therapeutic effects of curcumin are being evaluated in over 100 human clinical trials, targeting human diseases including but not limited to cancers, cardiovascular diseases, and inflammatory diseases such as arthritis and colitis.

A problem to develop curcumin-based therapeutics is its poor chemical stability: its half-life in aqueous buffer at physiological pH is only several minutes, leading to rapid formation of various degradation products, such as ferulic acid and feruloyl methane, and recently discovered bicyclopentadione derivatives of curcumin. Due to the short half-life of curcumin in aqueous buffer, it is difficult to determine whether the observed biological actions of curcumin are due to curcumin itself or its degradation products, hampering mechanistic understanding of curcumin biology. In addition, in part due to its rapid degradation at physiological pH curcumin has a poor pharmacokinetics profile, and high doses of curcumin were needed to exert its biological actions in vivo. Indeed, in the Phase IIα trial of colorectal cancer, curcumin had no effect at a dose of 2 grams/day, and only reduced colorectal cancer risks at a dose as high as 4 grams/day. Chronic intake of high-dose curcumin could cause some adverse effects and lead to patient withdrawal, limiting its therapeutic applications. Therefore, it is of practical importance to better understand the underlying mechanisms of curcumin degradation and develop novel strategies to increase its stability.

There is a need for increased chemical stability of phenolic compounds.

### BRIEF SUMMARY

The present teachings show that co-addition of a series of redox active antioxidants with diverse chemical structures dramatically increases the chemical stability of dietary phenolic compounds, enhanced their biological activities in cells, and boosted the circulating concentrations of these compounds in animal models. This leads to development of (1) more effective and safer dietary supplements, and (2) stabilized color compounds as food pigments.

In one or more embodiments, the method of these teachings for stabilizing dietary phenolic compounds in aqueous buffer includes adding to predetermined amount of a redox active antioxidant. In one instance, the predetermined amount ranges from about 19.5% to 312% of the amount of the dietary phenolic compound. In one instance, the dietary phenolic compounds include curcuminoids, Epigallocatechin gallate (EGCG), Catechin gallates. stilbenoids, and Resveratrol (3,5,4′-trihydroxy-trans-stilbene). In one instance, the redox active antioxidant is one of gallic acid, ascorbate or ascorbic acid (vitamin C), tert-butylhydroquinone (TBHQ), caffeic acid, rosmarinic acid, and Trolox (a water-soluble analog of vitamin E).

In one or more embodiments, the formulation of these teachings for stabilizing dietary phenolic compounds in aqueous buffer includes a first predetermined amount of a dietary phenolic compound and a second predetermined amount of a redox active antioxidant, a ratio of the second predetermined amount to the first predetermined amount selected such that stability of the dietary phenolic compound in aqueous solution is increased. In one instance, the second predetermined amount ranges from about 19.5% to 312% of the first predetermined amount of the dietary phenolic compound. In one instance, the dietary phenolic compounds include curcuminoids, Epigallocatechin gal late (EGCG), Catechin gallates, stilbenoids, and Resveratrol (3,5,4′-trihydroxy-trans-stilbene). In one instance, the redox active antioxidant is one of gallic acid, ascorbate or ascorbic acid (vitamin C), tert-butylhydroquinone (TBHQ), caffeic acid, rosmarinic acid, and Trolox (a water-soluble analog of vitamin E).

In the exemplary embodiment of curcumin, previous research has suggested two mechanisms to explain curcumin degradation in aqueous buffer (see supplemental figure FIGS. 6a, 6b): (1) hydroxyl ion mechanism, in which hydroxyl ion (OH⁻) attacks the carbonyl group of curcumin, generating break-down products such as ferulic acid and fertiloyl methane; and (2) phenolic radical mechanism, in which curcumin is first converted to a phenolic radical, which then migrates to the conjugated heptadienedione chain and initiates a chain reaction of curcumin degradation to generate cyclized compounds such as bicyclopentadione derivatives of curcumin. Recent research showed that the bicyclopentadione derivatives of curcumin, instead of ferulic acid and feruloyl methane, are the major degradation products, suggesting a critical role of the phenolic radical mechanism in curcumin degradation. This lead to the present teachings that redox active antioxidants increase the chemical stability and biological activity of curcumin, through suppressing the formation of curcumin phenolic radicals or by regenerating oxidized curcumin. The effects of a wide range of redox active antioxidants on the chemical stability and biological activity of curcumin have been systematically studied leading to the present teachings.

For a better understanding of the present teachings, together with other and further needs thereof, reference is made to the accompanying drawings and detailed description and its scope will be pointed out in the appended claims.

### DESCRIPTION OF THE DRAWINGS

FIGS. 1A1-1A2, 1B1-1B3 show that Curcumin rapidly degrades in phosphate buffer at physiological pH;

FIGS. 2A1, 2A2, 2B1, 2B2, 2B3 show results of the method of these teachings showing that antioxidants increase curcumin stability in phosphate buffer;

FIGS. 3A, 3B, 3C, 3D show results of the method of these teachings showing that antioxidants increase long-term stability of curcumin in phosphate buffer;

FIGS. 4A1-4A6 show results of these teachings showing that antioxidants increase anti-proliferative effect of curcumin in MC38 colon cancer cells;

FIGS. 5A, 5B show results of these teachings showing that co-administration of antioxidant increases plasma concentration of curcumin;

FIGS. 6A, 6B show two proposed mechanisms for curcumin degradation in aqueous buffer;

FIGS. 7A-7G show chemical structure of antioxidants using these teachings.

FIGS. 8A-8E show Dose-response effects of antioxidants on curcumin stability in phosphate buffer;

FIGS. 9A-9C show comparative stabilities of curcumin and its analog in phosphate buffer;

FIG. 10 shows results of these teachings that show that Curcumin rapidly degrades in serum-free basal DMEM medium, as assessed by HPLC analysis;

FIGS. 11A, 11B show preparation and analysis of curcumin, total degradation products of curcumin (TDP);

FIGS. 12A-12C show results of these teachings that show that Curcumin degradation products have weaker anti-proliferative effects than curcumin;

FIGS. 13A1-13A5, 13B1-13B5 show results of these teachings that show that Curcumin degradation products have weaker effects on cell cycle progression and apoptosis than curcumin;

FIGS. 14A, 14B1, 14B2, 14C1, 14C2 show results of these teachings that show that Curcumin degradation products have weaker anti-inflammatory effects than curcumin;

FIGS. 15A, 15B show results of these teachings that show that Curcumin degradation products have weaker inhibitory effects on NF-kB signaling than curcumin;

FIG. 16A shows the chemical structure of EGCG; and

FIG. 16B show results of these teachings that show that that antioxidants increase EGCG stability in phosphate buffer.

### DETAILED DESCRIPTION

The description is not to be taken in a limiting sense, but is made merely for the purpose of illustrating the general principles of these teachings, since the scope of these teachings is best defined by the appended claims.

The above illustrative and further embodiments are described below in conjunction with the following drawings, where specifically numbered components are described and will be appreciated to be thus described in all figures of the disclosure.

As used herein, the singular forms “a,” “an,” and “the” include the plural reference unless the context clearly dictates otherwise.

“Biological activity,” as used herein, includes modulating cell proliferation, modulating cell cycle progression and apoptosis in cancer cells, and inhibiting inflammatory responses and NF-kB signaling.

The present teachings show that co-addition of a series of redox active antioxidants with diverse chemical structures dramatically increases the chemical stability of dietary phenolic compounds, enhanced their biological activities in cells, and boosted the circulating concentrations of these compounds in animal models. This leads to development of (1) more effective and safer dietary supplements, and (2) stabilized color compounds as food pigments.

In one or more embodiments, the method of these teachings for stabilizing dietary phenolic compounds in aqueous buffer includes adding to predetermined amount of a redox active antioxidant. In one instance, the predetermined amount ranges from about 19.5% to 312% of the amount of the dietary phenolic compound. In one instance, the dietary phenolic compounds include curcuminoids. Epigallocatechin gallate (EGCG), Catechin gallates. stilbenoids, and Resveratrol (3,5,4′-trihydroxy-trans-stilhene). In one instance, the redox active antioxidant is one of gallic acid, ascorbate (the anion of ascorbic acid, vitamin C) or ascorbic acid, tert-butylhydroquinone (TBHQ), caffeic acid, rosmarinic acid, and Trolox (a water-soluble analog of vitamin E).

In one or more embodiments, the formulation of these teachings for stabilizing dietary phenolic compounds in aqueous buffer includes a first predetermined amount of a dietary phenolic compound and a second predetermined amount of a redox active antioxidant, a ratio of the second predetermined amount to the first predetermined amount selected such that stability of the dietary phenolic compound in aqueous solution is increased. In one instance, the second predetermined amount ranges from about 19.5% to 312% of the first predetermined amount of the dietary phenolic compound. In one instance, the dietary phenolic compounds include curcuminoids, Epigallocatechin gallate (EGCG), Catechin gallates, stilbenoids, and Resveratrol (3,5,4′-trihydroxy-trans-stilbene). In one instance, the redox active antioxidant is one of gallic acid, ascorbate or ascorbic acid (vitamin C), tert-butylhydroquinone (TBHQ), caffeic acid, rosmarinic acid, and Trolox (a water-soluble analog of vitamin E).

In order to further elucidate these teachings, and exemplary embodiment is presented. It should be noted that these teachings are not limited only to the exemplary embodiment.

Antioxidants, including gallic acid, ascorbate or ascorbic acid (vitamin C), tert-butylhydroquinone (TBHQ), caffeic acid, rosmarinic acid, Trolox (a water-soluble analog of vitamin E), and disodiumethylenediaminetetra acetic acid (EDTA), were purchased from Thermal Fisher Scientific (Waltham, Mass.) or Sigma-Aldrich (St. Louis, Mo.). HPLC solvents were purchased from Thermo Fisher Scientific (Waltham, Mass.).

Curcumin (>98% purity) was purchased from Thermal Fisher Scientific (Waltham, Mass.).

Since many commercial samples of curcumin contain other curcuminoids, pure curcumin was prepared by chemical synthesis as described. Briefly, vanillin (3.04 g) and tributyl borate (10.8 mL) were added into a solution of acetylacetone (1.03 mL) and boric anhydride (0.35 g) in anhydrous ethyl acetate at 50° C., then n-butylamine (0.4 mL) dissolved in ethyl acetate was drop-wise added and the reaction was stirred overnight. Hydrochloric acid (1N, 30 mL) was added and stirred for another 30 min. The reaction mixture was extracted with ethyl acetate, and pure curcumin was obtained after methanol re-crystallization. Dimethoxy curcumin [1,7-bis(3,4-dimethoxyphenyl)-1,6-heptadiene-3,5-dione] was synthesized using the same strategy of curcumin synthesis, except vanillin was replaced with 3,4-dimethoxybenzaldehyde. The structure and purity of synthesized curcumin and its analog were confirmed by NMR, TLC and HPLC, reported previously.

To assess curcumin stability using UV spectroscopy, a 25 μM curcumin solution in 0.1 M phosphate buffer (2.5 g/L KH2PO4 and 11.5 g/L Na2HPO4, pH=7.4) was freshly prepared in UV quartz cuvettes, then UV spectrum (110-550 nm) was continuously recorded at different time points. To study the effects of antioxidants on curcumin stability in the buffer, a phosphate solution of 25 μM curcumin, with or without antioxidant, was freshly prepared and its absorbance at 420 nm was continuously recorded using a plate reader (Molecular Devices, Sunnyvale, Calif.).

A 25 μM curcumin solution, with or without antioxidant, was freshly prepared in 0.1 M phosphate buffer (pH=7.4), curcumin concentration in the buffer was analyzed by HPLC on an Agilent 1100 HPLC system, using an Agilent TC-C18(2) column (4.6×250 mm, 5 μm) eluted with a mobile phase of 80% methanol with 0.1% acetic acid and 20% water with 0.1% acetic acid, flow rate=1 mL/min, detection wavelength at 420 nm.

MC38 colon cancer cells (a gift from Prof. Ajit Varki at the University of California, San Diego) were plated into 96-well plates (6,000 cells per well) in 100 μL Dulbecco's Modified Eagle Medium (DMEM, purchased from Lonza, Allendale, N.J.) fortified with 10% Fetal Bovine Serum (PBS, purchased from Coming Inc., Corning, N.Y.) and allowed to attach overnight. The cells were then treated with curcumin, with or without redox active antioxidants, in DMEM basal medium for 24 h. Cell proliferation was assessed by 3-(4,5-dimethylthiazol-2-yl)-2,5-diphenyltetrazolium bromide (MTT) assay, as described in Zhang, G.; Nitteranon, V.; Chan, L. Y.; Parkin, K. L. Glutathione conjugation attenuates biological activities of 6-dehydroshogaol from ginger. Food Chem. 140:1-8; 2013, which is incorporated by reference herein in its entirety and for all purposes.

The animal experiment was conducted in accordance with the protocols approved by the Institutional Animal Care and Use Committee (IACUC) of University of Massachusetts Amherst.

Briefly, 20 mg/kg curcumin, with or without 40 mg/kg TBHQ, dissolved in 50 μL of DMSO was intraperitoneally (i.p.) injected into 6-week-old male Swiss Webster mice. After 1 h, the mice were sacrificed to harvest the blood. The harvested blood was centrifuged at 1,200 g for 10 min at 4° C. to prepare the plasma fraction; then 200 μL plasma was immediately extracted with 400 μL ethyl acetate, the ethyl acetate extract was dried and reconstituted in methanol for HPLC analysis. The HPLC was performed on an Agilent 1100 HPLC system, using an Agilent TC-C18(2) column (4.6×250 mm, 5 μm), flow rate=1 mL/min, detection wavelength at 420 nm.

The mobile phase consisted methanol with 0.1% acetic acid (mobile phase B) and water with 0.1% acetic acid (mobile phase A). The extracted sample was eluted on a gradient starting with 65% B for 5 min, increasing to 75% B in 5 min and holding at 75% B for 5 min, decreasing to 65% B in 1 min and holding at 65% B for 4 min.

Group comparisons were carried out using one-way analysis of variance or Student t test. P values less than 0.05 were considered statistically significant.

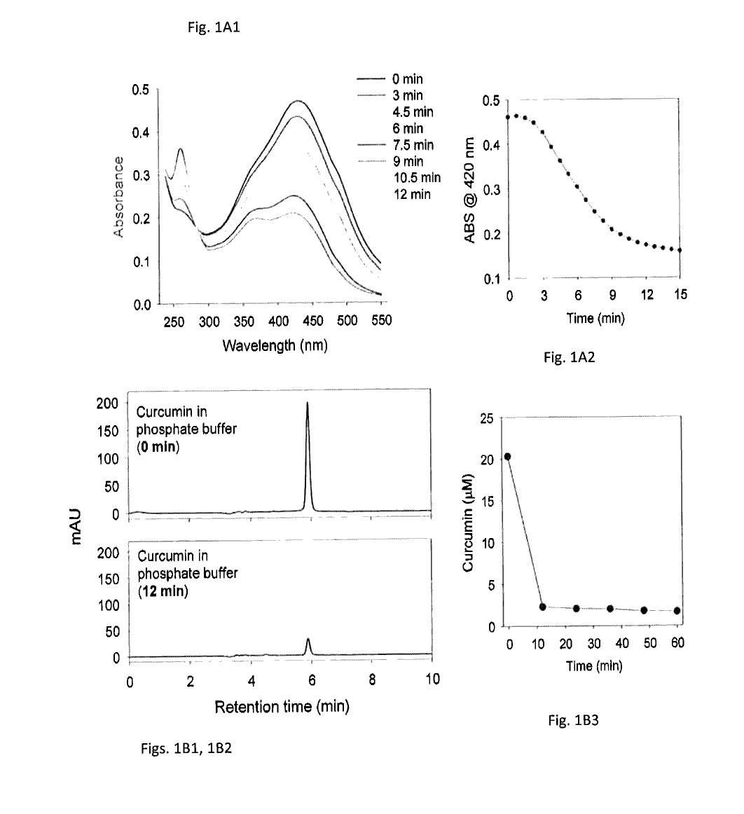

FIGS. 1A1, 1A2, 1B1, 1B2, 1B3 show that Curcumin rapidly degrades in phosphate buffer at physiological pH. (A) UV spectrum shows a rapid degradation of curcumin in phosphate buffer, with a time-dependent decrease of absorbance at 420 nm. (A1) representative UV spectrum of a 25 μM curcumin solution prepared in 0.1 M phosphate buffer (pH=7.4). (A2) quantification of absorbance at 420 nm, (B) HPLC analysis also shows a rapid degradation of curcumin in phosphate buffer. (B1, B2) representative HPLC chromatography of a 25 μM curcumin solution in 0.1 M phosphate buffer, detection wavelength at 420 nm. (B3) quantification of curcumin concentration in the phosphate buffer by using HPLC.

Consistent with previous studies, it has been found that curcumin has a poor stability in aqueous buffer at physiological pH. UV spectroscopy analysis showed that curcumin rapidly degraded in phosphate buffer (pH=7.4), with a time-dependent decrease of absorbance at λmax of curcumin (˜420 nm) (FIGS. 1A1, 1A2). This is consistent with previous studies, which showed that the degradation products of curcumin do not exhibit absorbance at 420 nm. To further validate the results obtained from spectroscopic analysis, reverse-phase HPLC was used to measure curcumin concentration in the phosphate buffer. HPLC analysis showed that after ˜12-min, incubation, there is an approximate 80-90% degradation of curcumin in the buffer, followed with a slower degradation of the remaining curcumin (FIGS. 1B1, 1B2, 1B3). Together, these results confirm the poor chemical stability of curcumin in aqueous buffer at physiological pH.

After the poor stability of curcumin in aqueous buffer had been established, the effects of antioxidants on curcumin stability were studied using a colorimetric assay and HPLC analysis. As shown in FIG. 1A2, curcumin degradation in the buffer is correlated with a decrease of absorbance at its λmax (˜420 nm); therefore, a 96-well-plate-based colorimetric assay was used to rapidly screen antioxidants on their effects to modulate curcumin stability.

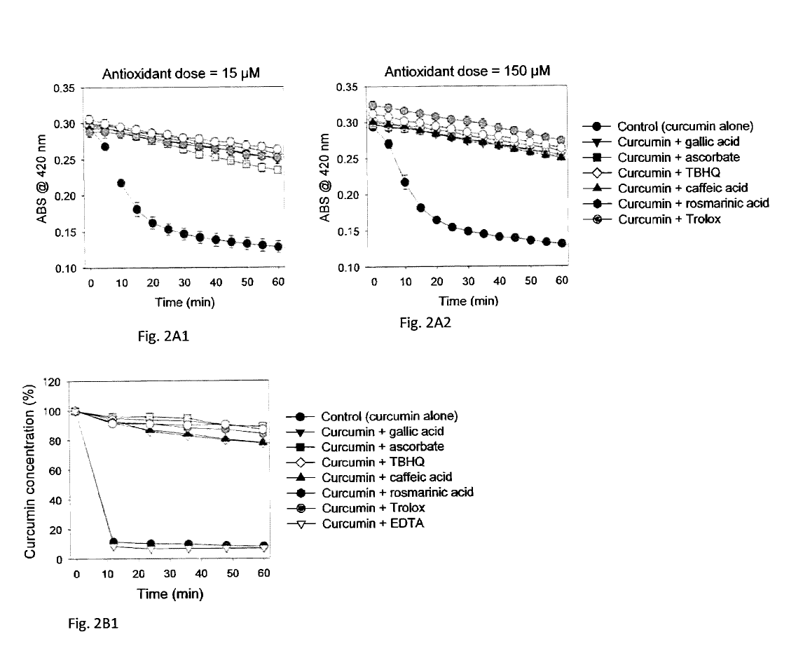

FIGS. 2A1, 2A2, 2B1, 2B2, 2B3 show results of the method of these teachings showing that antioxidants increase curcumin stability in phosphate buffer, (A) A colorimetric assay shows that co-addition of antioxidants (dose of antioxidants=15 and 1.50 μM) increases curcumin stability in phosphate buffer. (B) HPLC analysis confirms that these antioxidants (dose of antioxidant=10 μM), except EDTA, increased curcumin stability in phosphate buffer. (Left) quantification of curcumin concentration in the phosphate buffer by using HPLC, the results are expressed as percentage of curcumin concentration at a specific time point to that at time 0. (Right) representative HPLC chromatography, detection wavelength at 420 nm. The results are mean ±SD.

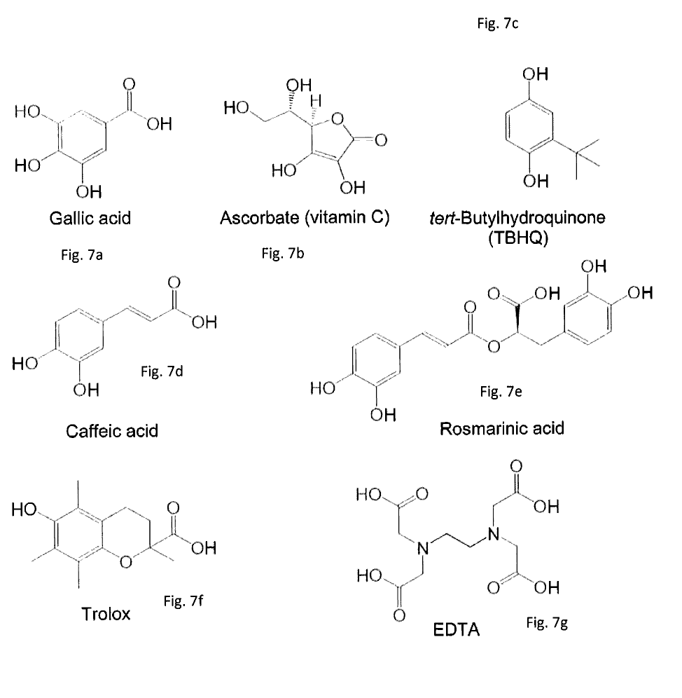

When incubated alone in phosphate buffer, curcumin rapidly degraded, while co-addition of a wide range of redox active antioxidants dramatically increased curcumin stability (FIG. 2A). These antioxidants possessed diverse chemical structures, including gallic acid, ascorbate (vitamin C), TBHQ, caffeic acid, rosmarinic acid, and. Trolox (a water-soluble analog of vitamin E) (see chemical structures of these antioxidants in figure FIGS. 7a-7g). Co-addition of these antioxidants, such as ascorbate or Trolox, did not change pH of the phosphate buffer (data not shown), indicating the protective effects were not due to altered pH.

Next HPLC analysis was performed to further validate the results obtained from the colorimetric assays. Consistent with the colorimetric assays, co-addition of above redox active antioxidants dramatically decreased curcumin degradation in the phosphate buffer within a 1-h incubation period (FIG. 2B), confirming the protective effects of antioxidants on curcumin stability. EDTA was also tested, which is a widely used metal-chelating antioxidant that does not have radical scavenging activity. The HPLC analysis showed that EDTA, even at a dose as high as 200 μM, had little protective effect on curcumin stability (FIG. 2A).

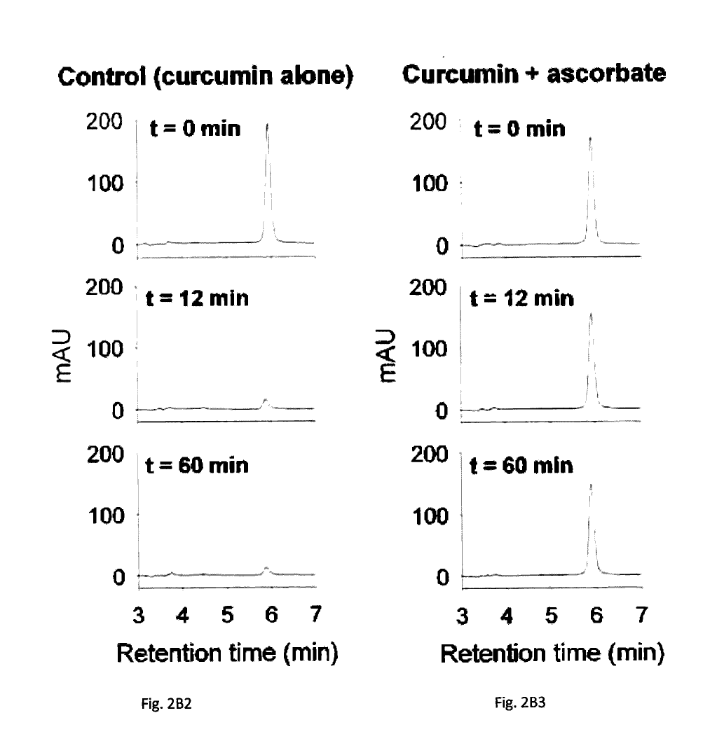

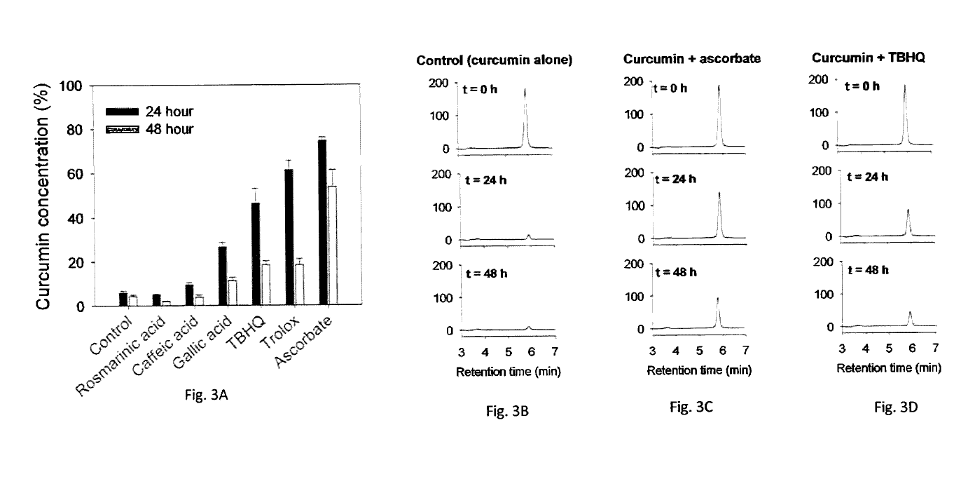

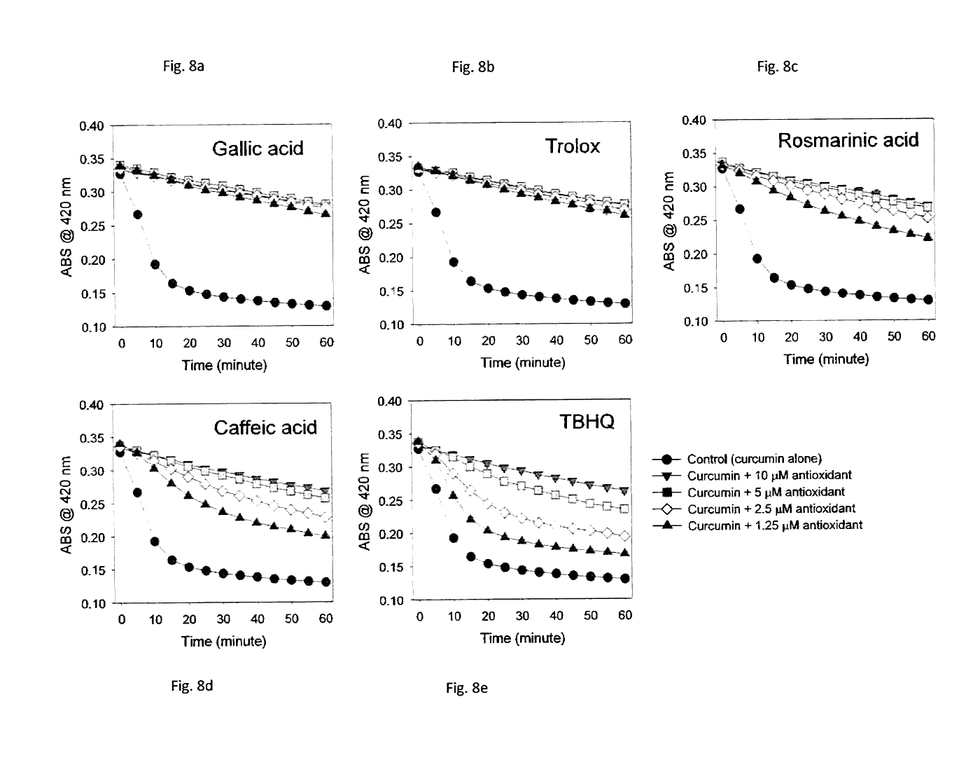

The dose-response effects of these antioxidants on curcumin stability, and the actions of antioxidants on long-term stability of curcumin were further characterized. The dose-response experiment showed that these redox active antioxidants significantly increased curcumin stability at doses as low as 1 μM (FIGS. 8a-8e), demonstrating potent protective effects of these antioxidants on curcumin stability. Regarding the long-term stability, co-addition of ascorbate significantly increased long-term stability of curcumin: after 24-h and 48-h incubation, there was respective ˜74.7% and ˜53.7% of curcumin remaining in the phosphate buffer. In contrast, when curcumin was incubated alone, there was only ˜5.9% and ˜4.2% curcumin remaining after 24-h and 48-h incubation, respectively (FIG. 3A).

FIGS. 3A, 3B-3D results of the method of these teachings showing that antioxidants increase long-term stability of curcumin in phosphate buffer. (FIG. 3A) quantification of curcumin concentration after 24-h and 48-h. incubations. To study the effects of antioxidants on long-term stability of curcumin, a 25 μM curcumin solution in 0.1 M phosphate buffer, with or without antioxidants (dose of antioxidant=150 μM), was freshly prepared, and the concentration of curcumin at different time point was analyzed by HPLC. The results are expressed as percentage of curcumin concentration at a specific time point to that at time 0. (FIGS. 3B-3D) representative HPLC chromatography. The results are mean ±SD.

Considering the half-life of curcumin in phosphate buffer was less than 10 minutes (see FIG. 1B3), co-addition of ascorbate caused a >200-fold increase of the half-life of curcumin. Besides ascorbate, other antioxidants, such as gallic acid, TBHQ and Trolox, also significantly increased long-term stability of curcumin (FIG. 3A), suggesting that co-administration of redox active antioxidants could be a practical strategy to enhance curcumin stability.

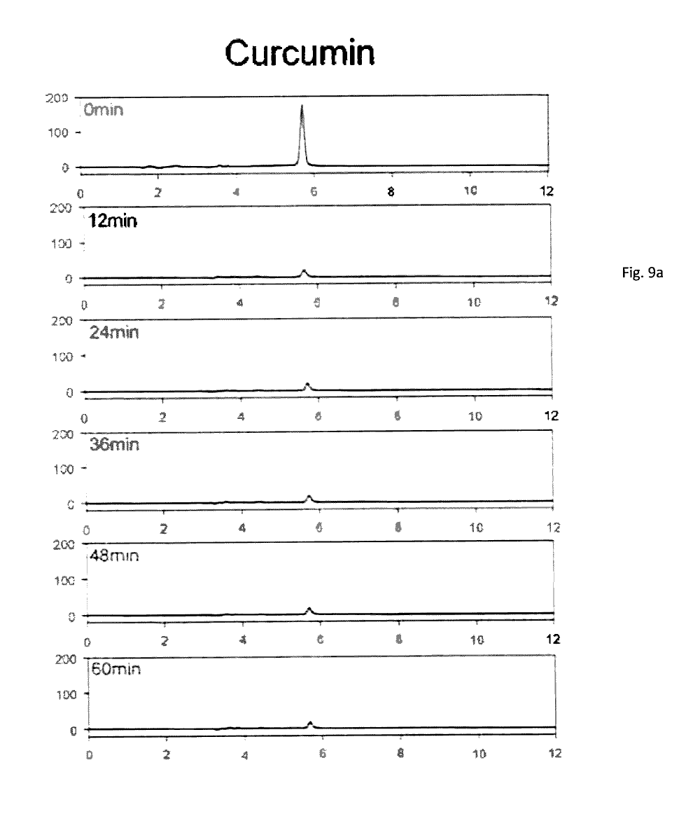

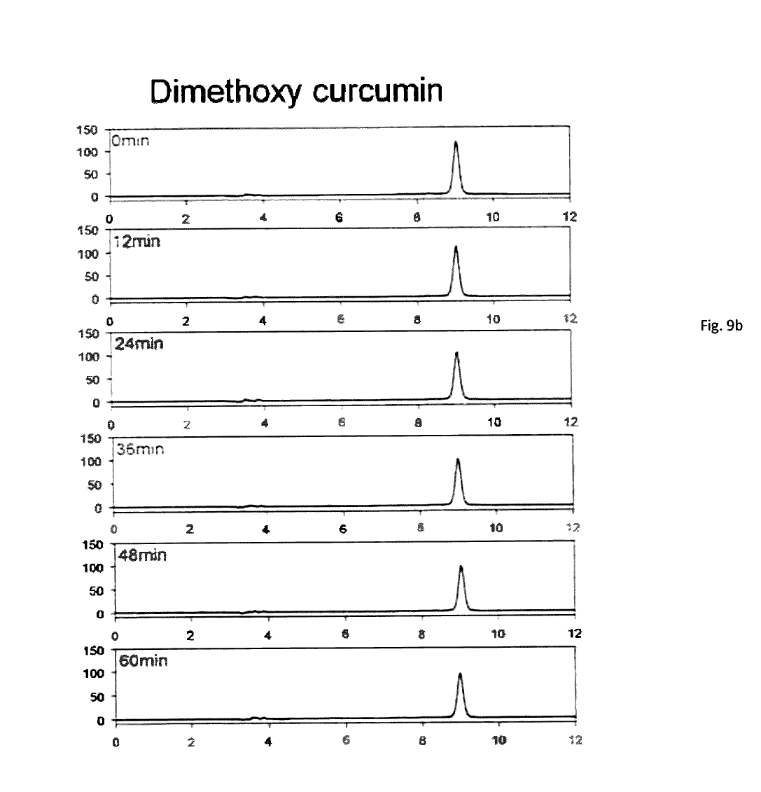

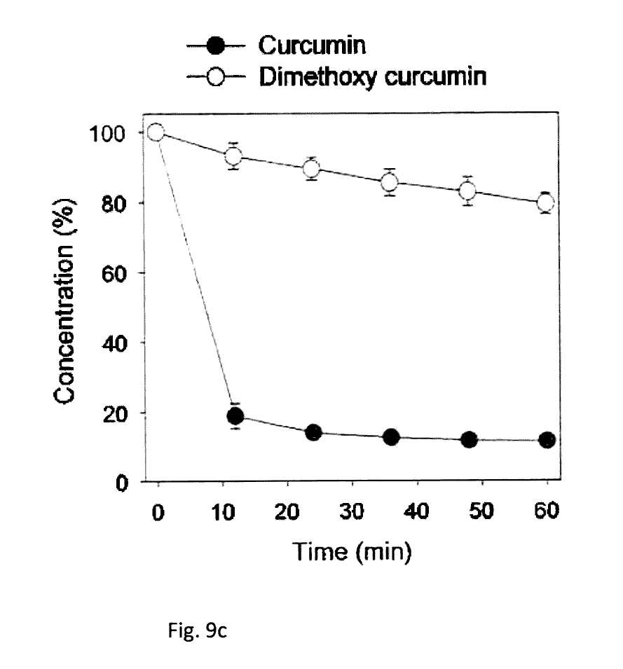

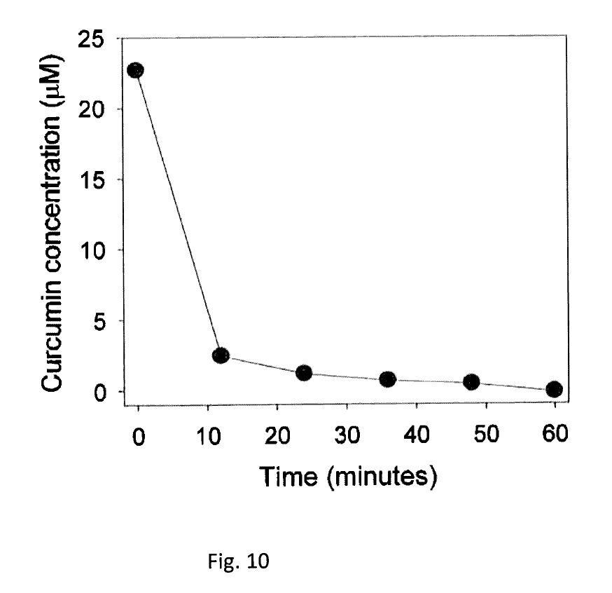

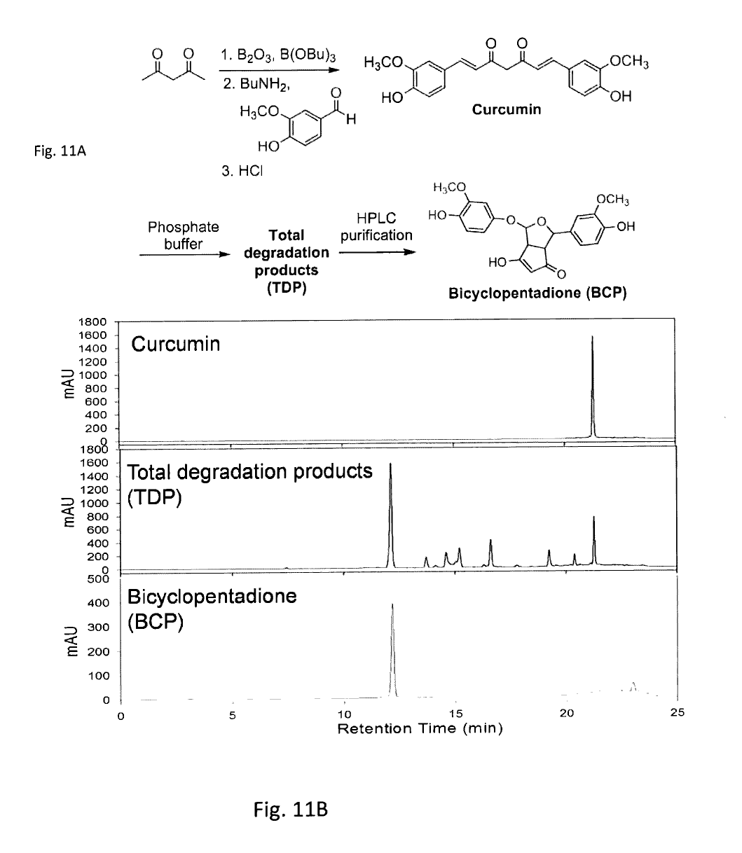

The results of these teachings above strongly suggest that the phenolic radical mechanism, instead of the hydroxyl ion mechanism, plays a critical role in mediating curcumin degradation. To further test which mechanism plays a major role, the stability of curcumin was compared with its structural analog dimethoxy curcumin (see chemical structures in FIGS. 9a-9c). FIGS. 9a-9c, show comparative stabilities of curcumin and its analog in phosphate buffer. FIGS. 9a, 9b show HPLC analysis shows that curcumin rapidly degrades, while dimethoxy cut-cumin is very stable. To compare their stabilities, 25 μM solution of each compound was freshly prepared in 0.1 M phosphate buffer (pH=7.4), then the concentration of each compound at different time point was analyzed by HPLC, detection wavelength at 420 nm. FIG. 9c shows quantification of concentration of curcumin and dimethoxy curcumin. The results are mean ±SD.

Compared with curcumin, dimethoxy curcumin does not have the radical-initiating phenolic groups. HPLC analysis showed that in phosphate buffer, curcumin rapidly degraded, while dimethoxy curcumin was very stable (FIGS. 9a-9c). This result further supports that the phenolic radical mechanism, instead of the hydroxyl ion mechanism, plays a major role in mediating curcumin degradation.

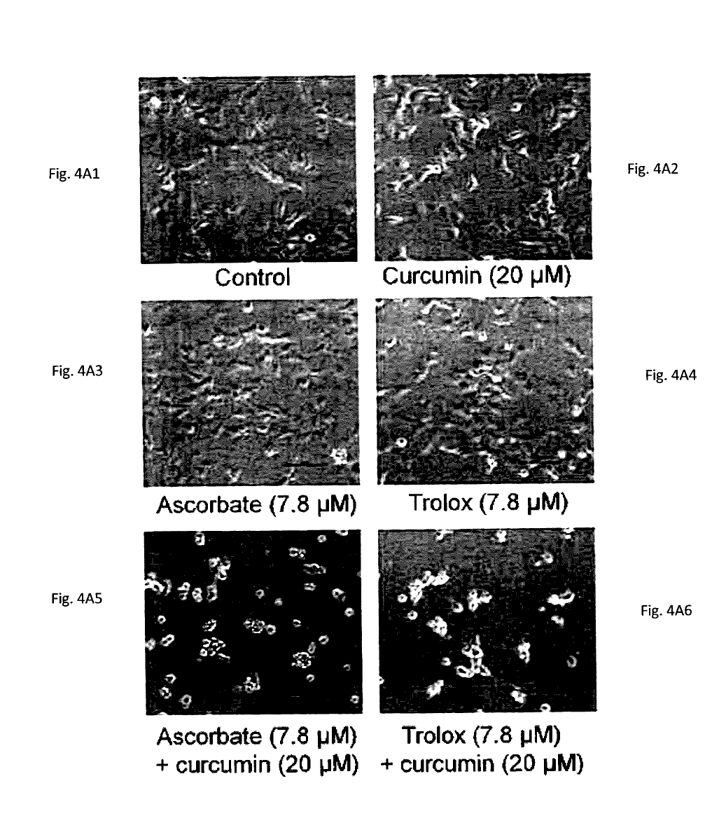

After it was demonstrated that redox active antioxidants stabilize curcumin in aqueous buffer, their effects on the biological activity of curcumin in cultured cells were tested. Cellular assays in serum-free basal medium were conducted, since curcumin rapidly degraded in the basal medium (supplemental figure FIG. S5). In MC38 colon cancer cells, treatment with 20 μM curcumin slightly suppressed cell proliferation, with a ˜20% inhibition of cell proliferation; and treatment with 3.9-62.5 μM ascorbate err Trolox alone had no effect on MC38 cell proliferation, FIGS. 4A1-4A6, 4B, 4C, 4D show results of these teachings showing that antioxidants increase anti-proliferative effect of curcumin in MC38 colon cancer cells. (A1-A6) Representative microscope images of the treated cells. Treatment with curcumin alone, or Trolox or ascorbate alone, had little effect on cell density or morphology; in contrast, their combination dramatically reduced cell density and changed cell morphology. (B) Quantification of cell proliferation of MC38 cells treated with curcumin (20 μM), or several different doses of ascorbate (3.9-62.5 μM), or a combination of curcumin and ascorbate, for 24 h. (C) Quantification of cell proliferation of MC38 cells treated with curcumin (20 μM), or several different doses of Trolox (3.9-62.5 μM), or a combination of curcumin and Trolox, for 24 h. (D) Quantification of cell proliferation of MC38 cells treated with curcumin (20 μM), or several different antioxidant (dose of each antioxidant=3.9 μM), or a combination of curcumin and antioxidant, for 24 h. The results are mean ±SD, The samples designated with different letters are statistically different (P <0.05).

In contrast, the combination of 20 μM curcumin with 3.9-62.5 μM ascorbate err Trolox dramatically suppressed cell proliferation, with a 52-74% and 42-70% inhibition of cell proliferation, respectively (FIG. 4A1-C). Besides ascorbate and Trolox, other antioxidants were also tested, and found all redox active antioxidants used in our study significantly increased anti-proliferative effect of curcumin in MC38 colon cancer cells (FIG. 4D). Together, these results demonstrate that redox active antioxidants enhance the biological activity of curcumin.

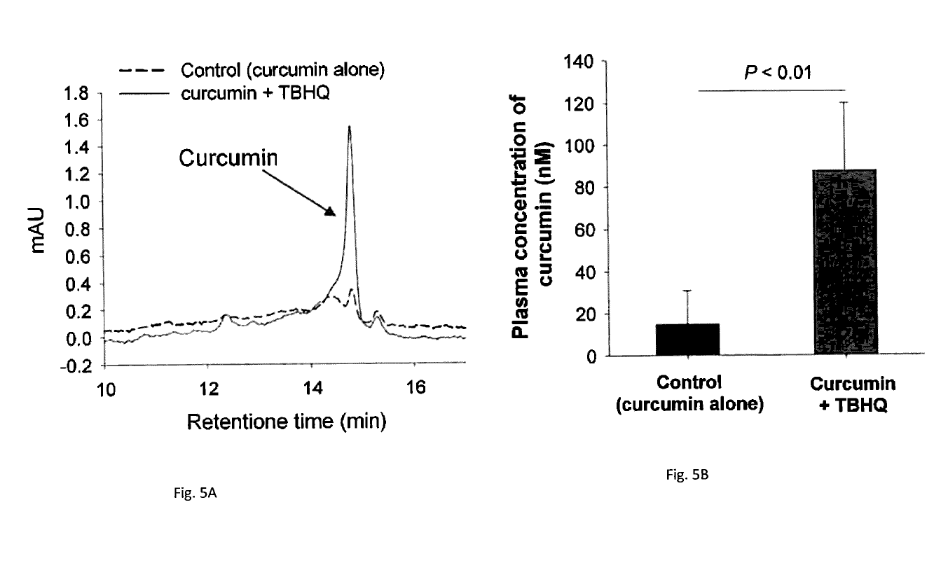

Finally, whether co-administration of antioxidant increased the circulating level of curcumin in animal models was tested. Consistent with previous studies, HPLC analysis showed that 1 h after i.p. injection of 20 mg/kg curcumin, the plasma concentration of curcumin was 14.8±16 nM. Co-administration of curcumin with TBHQ (dose=40 mg/kg), a redox active antioxidant widely used in food systems, increased the plasma concentration of curcumin to 87.6±32.3 nM (˜6-fold increase) (FIGS. 5A, 5B). FIGS. 5A, 5B show results of these teachings showing that co-administration of antioxidant increases plasma concentration of curcumin. FIG. 5A shows representative HPLC chromatography of the plasma from mice treated with control (20 mg/kg curcumin alone), or a combination of 20 mg/kg curcumin and 40 mg/kg tBHQ. FIG. 5B shows quantification of plasma concentration of curcumin, n=4 mice per group, the results are mean ±SD.

These results suggests that chemical degradation of curcumin could happen in vivo, and co-administration of antioxidant suppresses this degradation process and increase the circulating level of curcumin.

Here a finding is that a wide range of redox active antioxidants with diverse chemical structures significantly increased chemical stability and biological activity of curcumin. This finding suggests that a redox-dependent mechanism plays a major role in mediating curcumin degradation, and provides a novel and practical strategy to enhance the chemical stability and biological activity of curcumin.

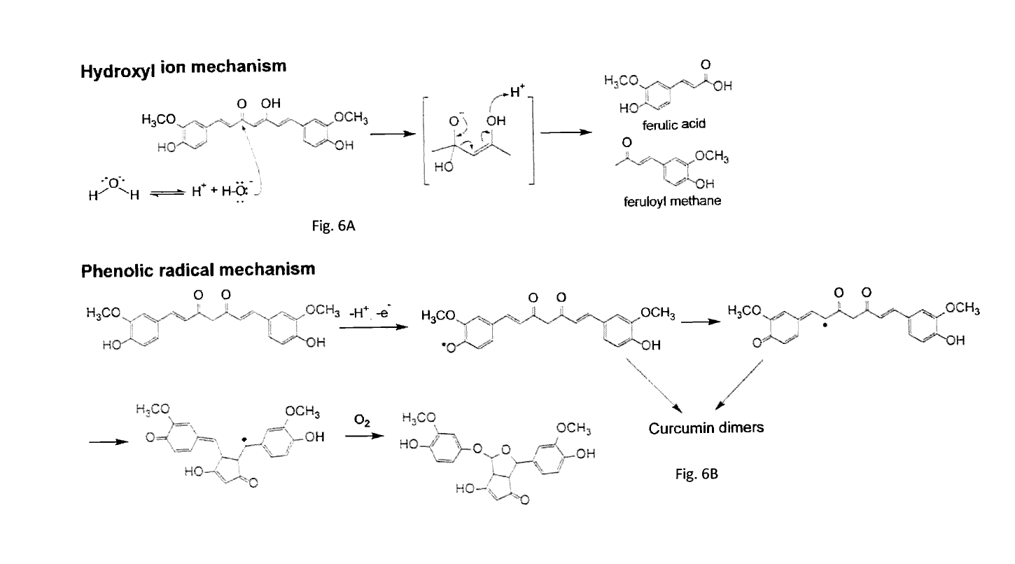

Previous research has suggested two mechanisms to explain curcumin degradation in aqueous buffer: hydroxyl ion mechanism and phenolic radical mechanism (see scheme in FIGS. 6a, 6b). FIGS. 6a, 6b show two proposed mechanisms for curcumin degradation in aqueous buffer. FIG. 6a shows a hydroxyl ion mechanism, in which hydroxyl ion (OH⁻) attacks the carbonyl group of curcumin, generating break-down products of curcumin such as ferulic acid and feruloyl methane. FIG. 6b shows a phenolic radical mechanism, in which curcumin is first converted to a phenolic radical, which then migrates to the conjugated heptadienedione chain, leading to formation of bicyclopentadione derivatives of curcumin and other degradation products.

The results shown here in using redox active antioxidants and curcumin analog strongly support that the phenolic radical mechanism, instead of the hydroxyl ion mechanism, plays a major role in mediating curcumin degradation. Together, the present teachings and previous investigations suggest that curcumin degradation in aqueous buffer could happen through a mechanism comparable to that of lipid peroxidation. The first step of curcumin degradation is hydrogen dissociation from the phenolic group to form a phenolic radical, which then migrates to the conjugated heptadienedione chain and leads to formation of cyclized compounds such as the recently identified bicyclopentadione derivatives of curcumin. The radical could be then transferred to another curcumin molecule, resulting in a chain reaction to cause a rapid and massive curcumin degradation. Both the phenolic group and the conjugated heptadienedione chain seem to play critical roles in curcumin degradation. Indeed, converting the radical-initiating phenolic (—OH) groups to methoxy groups (—OCH3), or reduction of the carbon-carbon double bonds in the conjugated heptadienedione chain, significantly increased curcumin stability (see FIGS. 9a-9c and Pan, M. H.; Huang, T. M.; Lin, J. K. Biotransformation of curcumin through reduction and glucuronidation in mice. Drug Metab Dispos 27:486-494; 1999, which is incorporated by reference herein in its entirety and for all purposes). The redox active antioxidants increase chemical stability of curcumin, at least in part, through directly suppressing formation of the curcumin phenolic radical. The redox active antioxidants used herein have been shown to have lower hydrogen dissociation energies compared with that of the phenolic group of curcumin. Therefore, these antioxidants can efficiently reduce the curcumin phenolic radicals, and terminate chain reaction of curcumin degradation. Together, these results suggest a redox-dependent mechanism as a major mechanism to mediate curcumin degradation. Regarding the effects of antioxidants on biological activities of curcumin, many previous studies have shown that co-addition of millimolar (mM) sulfhydryl antioxidants such as glutathione (GSH) and N-acetyl cysteine (NAC, a boosting agent of intracellular GSH) attenuated biological activities of curcumin in cultured cells. However, curcumin is highly sulfhydryl-reactive: it rapidly reacts with GSH to form curcumin-GSH adducts through a base-catalyzed Michael reaction or through a GSH S-transferase-catalyzed enzymatic process. Previous studies have shown that the resulting curcumin-GSH adduct is inactive or less-active in many cellular assays. Therefore, the results obtained from using GSH and NAC may actually reflect a GSH-dependent metabolism, but not the redox mechanisms of curcumin. Here it is shown that co-administration of low-micromolar (μM) doses of a wide range of redox active antioxidants, such as gallic acid, ascorbate, TBHQ, caffeic acid, rosmarinic acid, and Trolox, significantly increased anti-proliferative effects of curcumin in MC38 colon cancer cells (FIGS. 4A1-4A8, 4B, 4C, 4D). These antioxidants increase the biological activities of curcumin through increasing curcumin stability in the cell culture system.

Until now, it remained unclear whether the observed biological activities of curcumin are derived from curcumin itself or its degradation products.

The biological activities of curcumin degradation products, including its total degradation products (a mixture containing all stable degradation products of curcumin) and bicyclopentadione (a dominant stable degradation compound of curcumin) are studied herein. Consistent with previous studies, curcumin potently modulated cell proliferation, cell cycle progression and apoptosis in MC38 colon cancer cells, and inhibited lipopolysaccharide (LPS)-induced inflammatory responses and NF-kB signaling in RAW 264.7 macrophage cells. In contrast, neither the total degradation products of curcumin nor bicyclopentadione had such effects. Together, these results suggest that the stable chemical degradation products of curcumin are not likely to play a major roe in mediating the biological activities of curcumin,

Curcumin was chemically synthesized, since most commercial samples of curcumin contain other curcuminoids such as demethoxycurcumin and bisdemethoxycurcumin. The purity of the synthesized curcumin was >99% as assessed by HPLC and NMR. To study the biological activities of curcumin degradation products, the total degradation products of curcumin (TDP), which was a mixture containing all stable degradation products of curcumin after incubating curcumin in phosphate buffer, and BCP, which is was the most abundant degradation compound. of curcumin and was purified from TDP by HPLC, were prepared (FIGS. 11A, 11B).

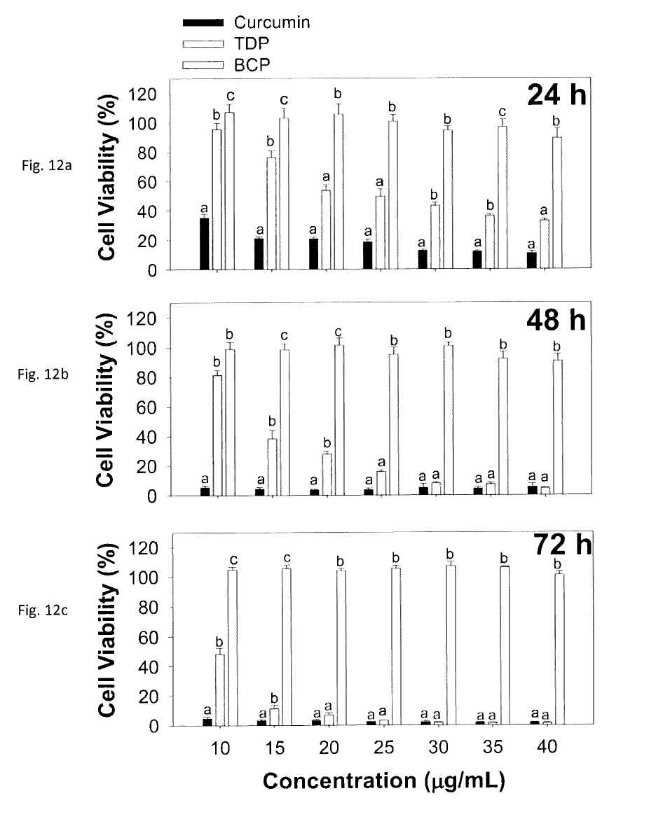

The effects of curcumin, TDP, and BCP on cancer cell proliferation were compared. Curcumin potently inhibited MC38 colon cancer cell proliferation in a dose- and time-dependent manner: at a dose of 10 μg/mL, curcumin inhibited ˜60% of MC38 proliferation after 24 h treatment, and inhibited >95% of MC38 proliferation after 48-72 h treatment (FIG. 2). This is consistent with previous studies which showed potent anti-proliferative effects of curcumin in various cancer cells. In contrast to the potent action of curcumin, the degradation products of curcumin, including TDP and BCP, had dramatically reduced anti-proliferative effects. For example, TI) at a dose of 10 μg/mL had no inhibitory effect on MC38 proliferation after 24 h treatment, and only inhibited ˜20% of cell proliferation after 48-72 h treatment. BCP was completely inactive to inhibit cancer cell proliferation in the tested dose range (10-40 μg/mL) after 24-72 h treatment (FIGS. 12a-12c). Together, these studies showed that curcumin, but not its stable autoxidation products, inhibited cancer cell proliferation.

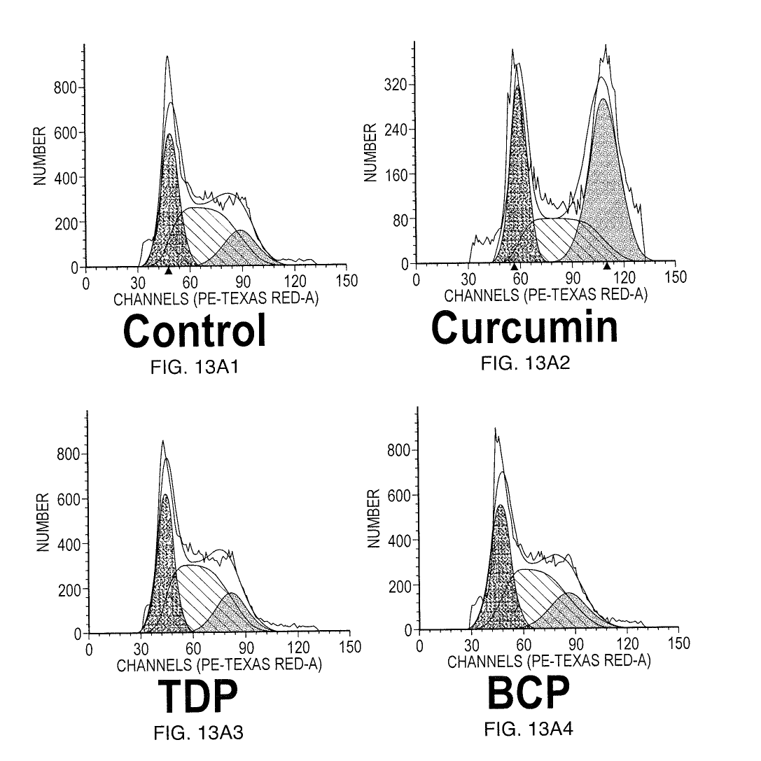

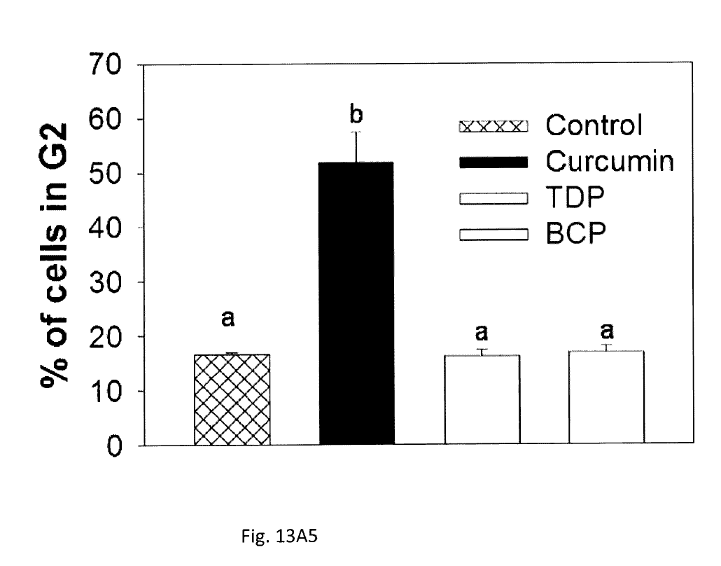

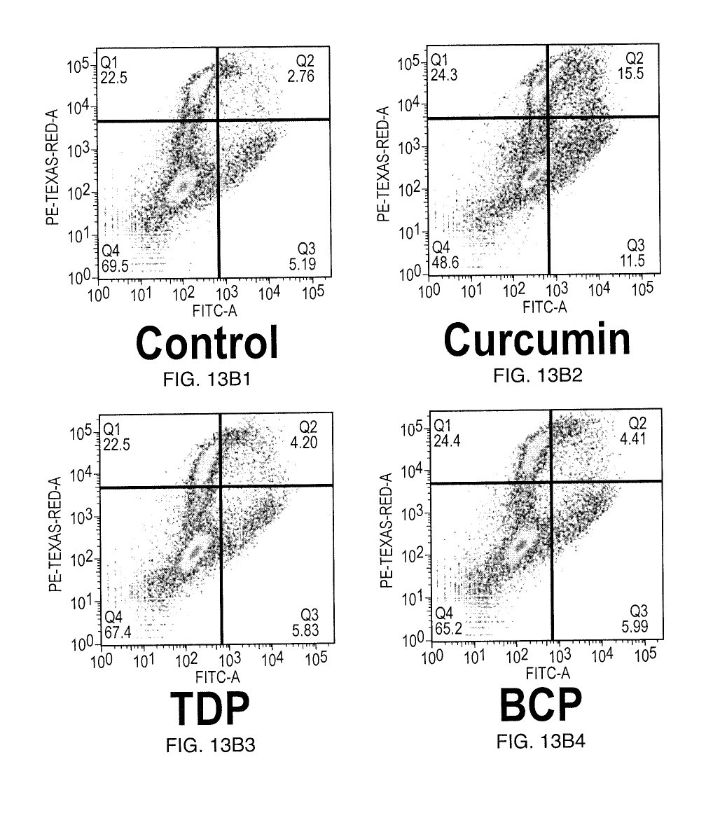

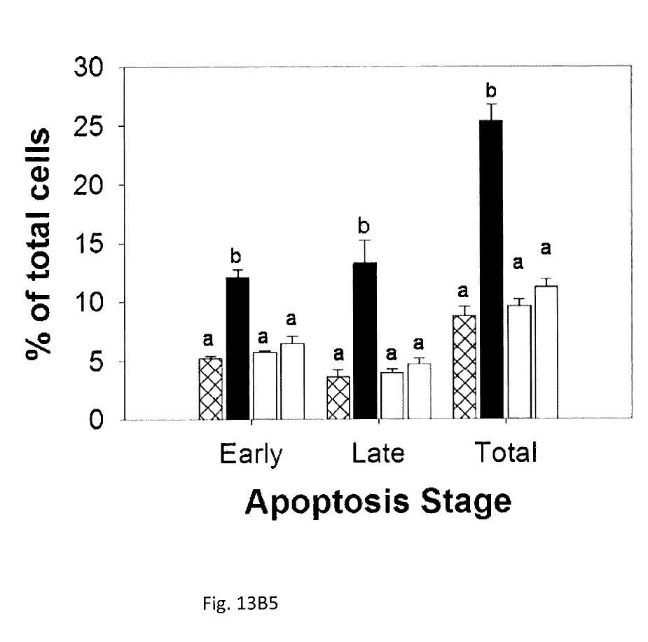

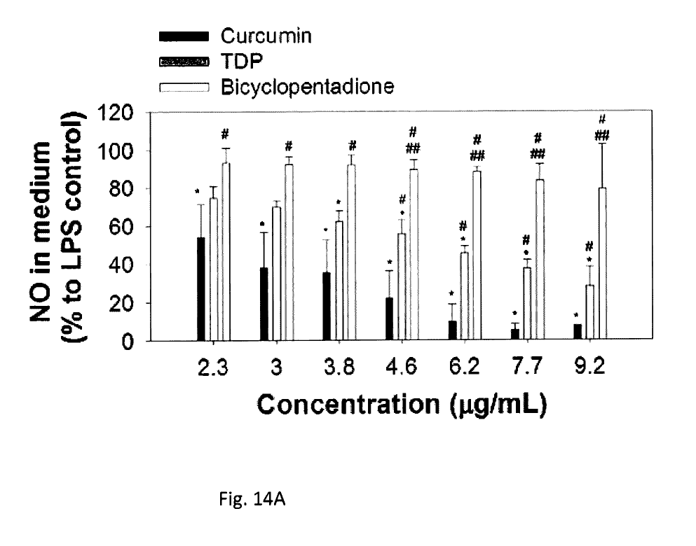

The effects of curcumin, TDP, and BCP on cell cycle progression and apoptosis, which are two critical processes involved in regulating cell proliferation, were further compared. After 24 hour treatment, curcumin significantly induced G2 cell cycle arrest and apoptosis in MC38 colon cancer cells; in contrast, neither TDP nor BCP had such effects (FIGS. 13A1-13A5, 13B1-13B5). These results further support that curcumin, but not its degradation products, modulated cancer cell proliferation and associated cellular responses.

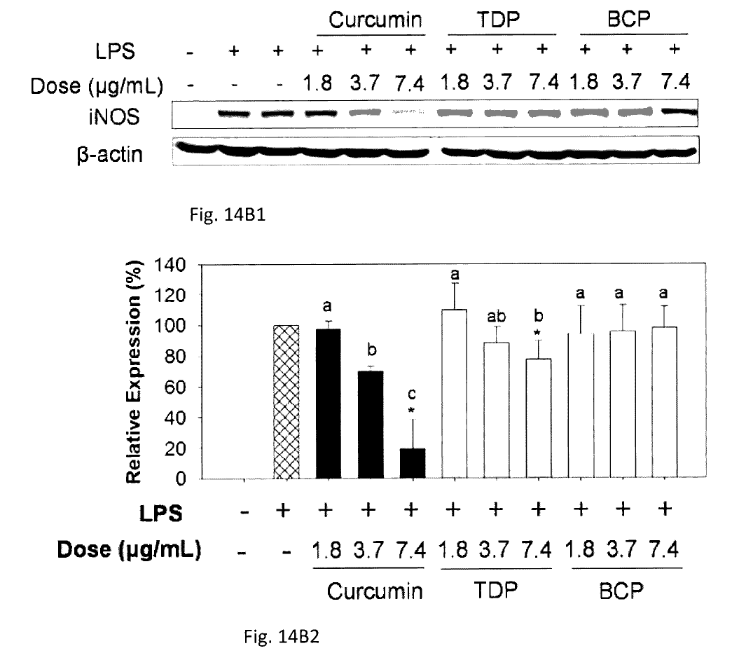

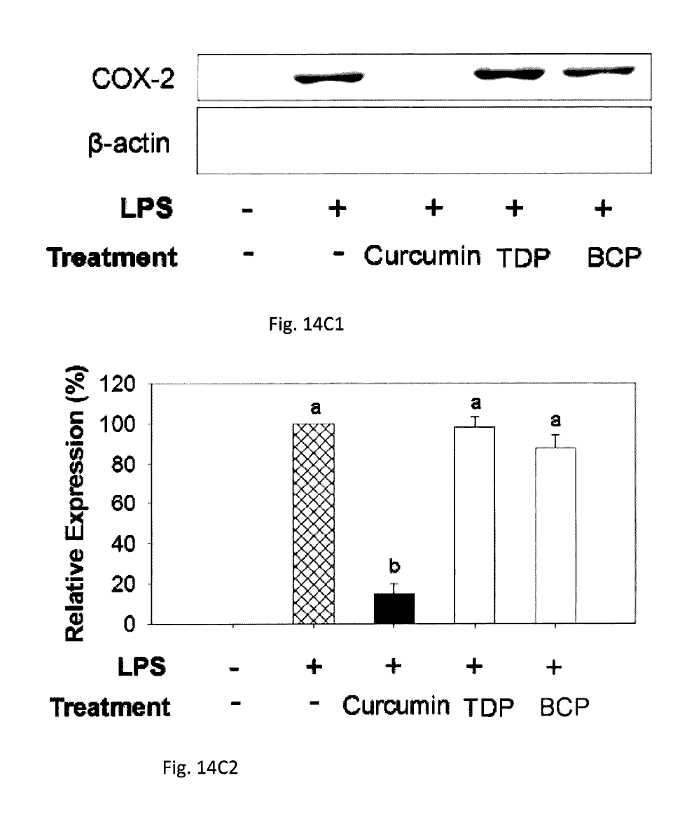

The effects of curcumin, TDP, and BCP on LPS-induced inflammatory responses in RAW 264.7 macrophage cells were compared. Consistent with previous studies, curcumin inhibited LPS-induced NO production in a dose-dependent manner. In contrast, TDP had a much weaker inhibitory effect and BCP had no effect (FIG. 14A). Consistent with the NO result, curcumin dose-dependently inhibited LPS-induced expression of iNOS, which is the major enzyme involved in production of NO; while TOP or BCP had no effect (FIGS. 14B1, 14B2). In these teachings, it has been also found that curcumin, but not its degradation products, inhibited LPS-induced expression of inflammatory protein COX-2 (FIG. 14C1, 14C2), Together, these results showed that curcumin, but not its degradation products, have anti-inflammatory effects.

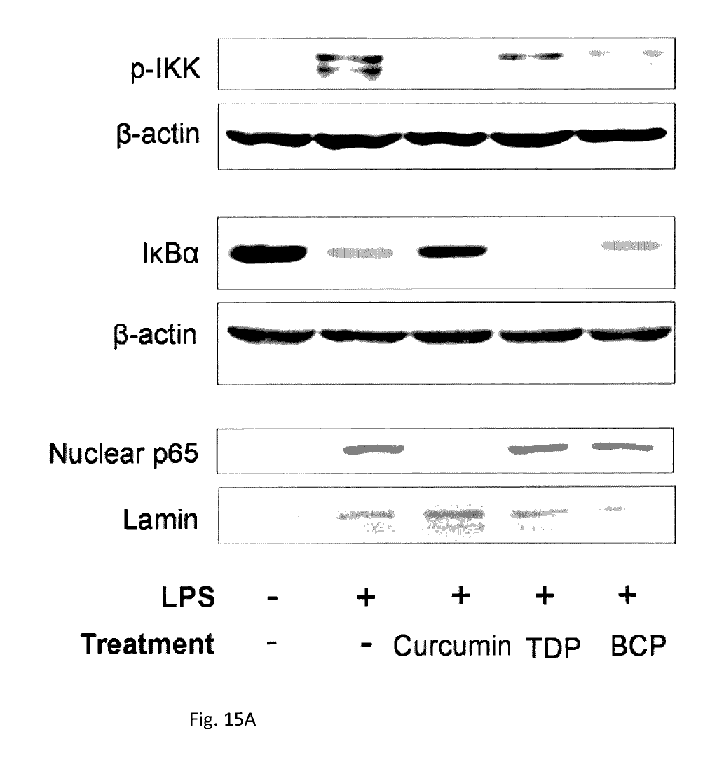

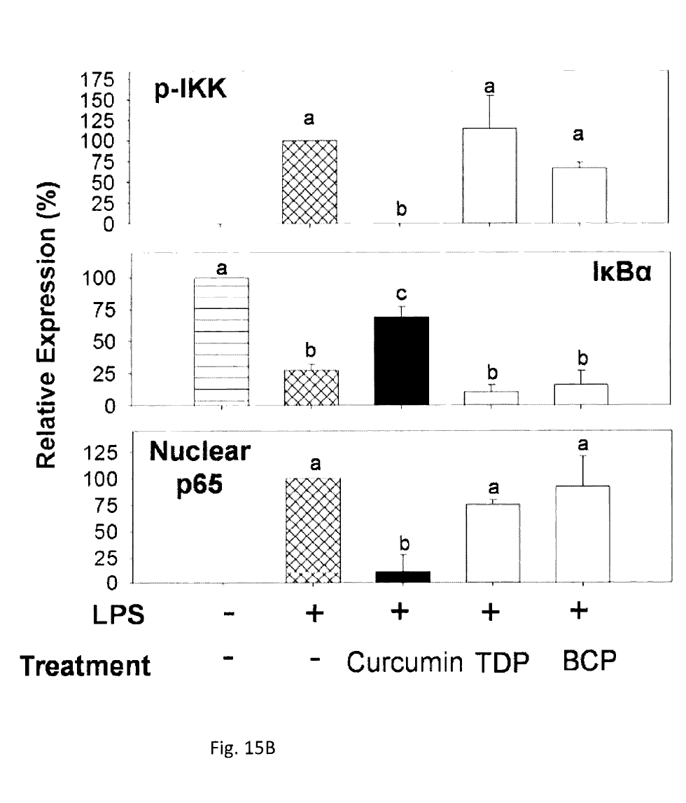

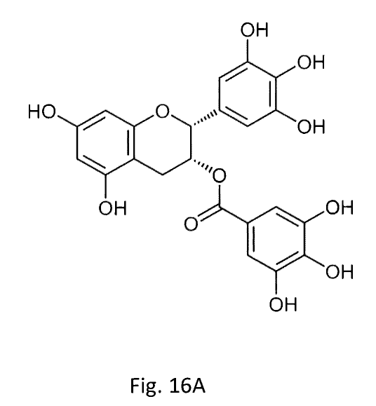

The effects of curcumin, TDP, and BCP on LPS-induced NF-kB signaling in RAW 264.7 macrophage cells were compared. Consistent with previous studies, curcumin inhibited LPS-induced IKK phosphorylation, IκBα degradation, and nuclear translocation of p65, demonstrating that curcumin inhibited LPS-induced activation of NF-kB signaling. In contrast, neither TDP nor BCP had such inhibitory effect (FIGS. 15A, 15B). Together, these results further suggest that curcumin, but not its degradation products, have anti-inflammatory effects.

Due to the rapid degradation of curcumin at physiological pH, recent research suggests that the resulted degradation products could contribute to the observed biological activities of curcumin. Here the finding is that compared with curcumin, the stable degradation products of curcumin such as TDP and BCP have dramatically reduced biological effects in vitro, suggesting that these degradation products are not likely to play a major role in mediating the biological activities of curcumin.

Here the results indicate that curcumin, rather than its degradation products, is largely responsible for the observed biological activities of curcumin; therefore, the redox active antioxidants which stabilize curcumin enhanced its biological actions.

Curcumin is a promising dietary compound for disease prevention and/or treatment. However, a major barrier to develop curcumin-based therapeutics is its poor pharmacokinetics profile in vivo. Indeed, a previous human study has shown that after a single oral dose of 10 or 12 grams of curcumin, free form of curcumin was barely detected in human plasma. High doses of curcumin were required to exert its health-promoting biological activities in animal and human studies. In a Phase IIα trial of colorectal cancer, curcumin had no effect at a dose of 2 grams/day; and only inhibited aberrant crypt foci at a dose as high as 4 grams/day. Chronic consumption of high-dose curcumin could cause some adverse effects and therefore limit its therapeutic applications. Herein it has been shown that co-administration of antioxidant TBHQ significantly increased circulating level of curcumin in animal models (FIGS. 5A, 5B). This result indicates that chemical degradation of curcumin could also happen in vivo, and redox active antioxidants could increase pharmacokinetics profiles of curcumin through suppressing the chemical degradation of curcumin. Consistent with the findings of these teachings, previous studies have shown that co-administration of redox active antioxidants, such as ascorbate (vitamin C), enhanced biological activities of curcumin in several different disease models in vivo (Paturnraj, S.; Wongeakin, N.; Sridulyakul, P.; Jariyapongskul, A.; Futrakul, N.; Bunnag, S. Combined effects of curcumin and vitamin C to protect endothelial dysfunction in the iris tissue of STZ-induced diabetic rats. Clin. Hemorheol. Microcirc. 35:481-489; 2006; Tarasub, N.; Junseecha, T.; Tarasub, C.; Na Ayutthaya, W. D. Protective Effects of Curcumin, Vitamin C, or their Combination on Cadmium-Induced Hepatotoxicity. J Basic Clin Pharm 3:273-281; 2012, both of which are incorporated by reference herein in their entirety and for all purposes), For example, in a Cadmium-induced hepatotoxicity model, administration of 200-400 mg/kg curcumin alone or 400 mg/kg ascorbate alone had no effect, while their combination dramatically inhibited Cadmium-induced hepatotoxicity in rats. It remains to decide whether ascorbate interact with curcumin through enhancing the chemical stability and circulating level of curcumin in vivo. Together, these studies suggest that co-administration of redox active antioxidants could serve as a practical strategy to enhance pharmacokinetics profiles and biological activities of curcumin.

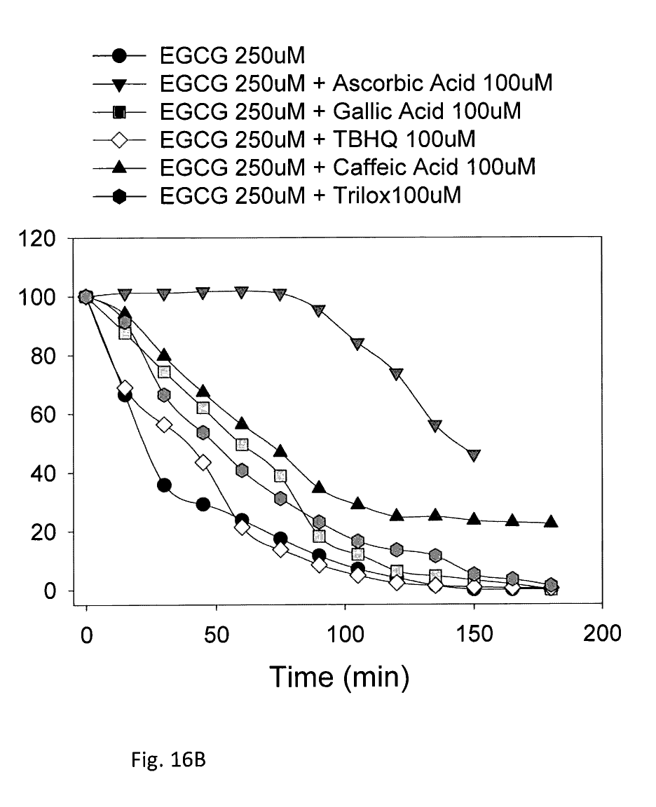

A 250 μM EGCG solution in a phosphate buffer (2.5 g/L KH2PO4 and 11.5 g/L Na2HPO4, pH=7.4) was prepared. FIG. 16A shows the chemical structure of EGCG. FIG. 16B shows HPLC analysis that confirms that the antioxidants (dose of antioxidant=100 μM), except perhaps gallic acid, increased curcumin stability in phosphate buffer.

In summary, these teachings show that a wide range of redox active antioxidants dramatically increased chemical stability and biological activity of curcumin in aqueous buffer, cultured cells and animal models. This further supports a previously proposed phenolic radical mechanism as a major mechanism that mediates curcumin degradation and provides a promising strategy to enhance the stability and biological activity of curcumin.

Although these teachings have been described with respect to various embodiments, it should be realized these teachings are also capable of a wide variety of further and other embodiments within the spirit and scope of the appended claims.

What is claimed is:

1. A method for stabilizing dietary phenolic compounds in solution in aqueous buffer, the method comprising adding a predetermined amount of a redox active antioxidant to the solution. The method of claim 1 wherein the predetermined amount is between about 19.5% to about 312% of an amount of a dietary phenolic compound.

3. The method of claim I wherein a dietary phenolic compound comprises at least one of curcuminoids, Epigallocatechin gallate (EGCG), Catechin gallates, stilbenoids, or Resveratrol (3,5,4′-trihydroxy-trans-stilbene).

4. The method of claim 1 wherein the redox active antioxidant comprises at least one of gallic acid, ascorbate, ascorbic acid (vitamin C), tert-butylhydroquinone (TBHQ), caffeic acid, rosmarinic acid, or Trolox (a water-soluble analog of vitamin E).

5. The method of claim 3 wherein the dietary phenolic compound comprises curcumin.

6. The method of claim 3 wherein the dietary phenolic compound comprises EGCG.

7. A formulation for stabilizing dietary phenolic compounds in aqueous buffer, the formulation comprising:
a first predetermined amount of a dietary phenolic compound; and
a second predetermined amount of a redox active antioxidant;
wherein a ratio of the second predetermined amount to the first predetermined amount is selected such that stability of the dietary phenolic compound in aqueous solution is increased.

8. The formulation of claim 7 wherein the second predetermined amount is between about 19.5% to about 312% of the first predetermined amount.

9. The formulation of claim 7 wherein a dietary phenolic compound comprises at least one of curcuminoids, Epigallocatechin gallate (EGCG), Catechin gallates, stilbenoids, or Resveratrol (3,5,4′-trihydroxy-trans-stilbene).

10. The formulation of claim 7 wherein the redox active antioxidant comprises at least one of gallic acid, ascorbate, ascorbic acid (vitamin C), tert-butylhydroquinone (TBHQ), caffeic acid, rosmarinic acid, or Trolox (a water-soluble analog of vitamin E).

11. The formulation of claim 10 wherein the dietary phenolic compound comprises curcumin.

12. The formulation of claim 10 wherein the dietary phenolic compound comprises EGCG.

13. The formulation of claim 12 wherein the redox active antioxidant is ascorbic acid.

14. A method for increasing biological activity of curcumin, the method comprising stabilizing curcumin in solution in an aqueous buffer;
wherein stabilizing curcumin comprises adding a predetermined amount of a redox active antioxidant to the solution; and
wherein stabilizing curcumin enhances biological activity.

15. The method of claim 14 wherein the redox active antioxidant comprises at least one of gallic acid, ascorbate, ascorbic acid (vitamin C), tert-butylhydroquinone (TBHQ), caffeic acid, rosmarinic acid, or Trolox (a water-soluble analog of vitamin E).

16. The method of claim 14 wherein the predetermined amount is between about 19.5% to about 312% of an amount of curcumin in the solution.
